(19) United States
(12) Patent Application Publication
(10) Pub. No.: US 2004/0107963 A1
(43) Pub. Date: Jun. 10, 2004

(54) Device and method for deagglomeration of powder for inhalation

(75) Inventors: Warren Finlay, Edmonton (CA); Zhaolin Wang, Edmonton, CA (US)

Correspondence Address: NIXON PEABODY, LLP, 401 9TH STREET, NW, SUITE 900, WASINGTON, DC, 20004-2128, US

(73) Assignee: THE GOVERNORS OF THE UNIVERSITY OF ALBERTA, Edmonton (CA)

(21) Appl. No.: 10/724,774

(22) Filed: Dec. 2, 2003

Related U.S. Application Data
(60) Provisional application No. 60/430,085, filed on Dec. 2, 2002.

(51) Int. Cl. A61M 15/00

(52) U.S. Cl. 128/203.15

(57) ABSTRACT

A device and method for deagglomerating powder agglomerates for inhalation. The device includes an inlet connected to a chamber and to a powder source for supplying the chamber with powder agglomerates and a flow of gas that define a swirling fluid flow inside the chamber. The device also includes an outlet connected to the chamber for inhalation such that the swirling fluid flow in the chamber can exit from the chamber as a longitudinal fluid flow that is directed along a longitudinal axis of the outlet, and a secondary fluid flow that is directed away from the longitudinal axis of the outlet. A mesh in the outlet prevents powder agglomerates above a predetermined size from traversing the mesh, and reduces the secondary fluid flow relative to the longitudinal fluid flow exiting from the chamber to thereby reduce powder deposition in a mouth and throat of a user.

### FIELD OF THE INVENTION

The present invention generally relates to a device and method for deagglomeration of powder agglomerates into finer powder particles for inhalation.

### BACKGROUND OF THE INVENTION

Dry powder inhalers are devices used to supply medication in the form of powder particles, which are typically inhaled by patients in the treatment of lung diseases, such as asthma and bronchitis. It is often required that the powders be fine, i.e., agglomerates of powder particles must be below given sizes. For instance, powders that are used in drug inhalers must be fine to avoid impaction in the mouth and throat of the user, i.e., the powder agglomerates must be below predetermined sizes to flow through the mouth and throat and reach the lungs when carried by an inspiratory flow.

Interparticle forces are the main reason for agglomeration of powder particles. Principal forces leading to deagglomeration are unclear. Particle deagglomeration can be caused by a variety of mechanisms, including creating a relative motion between the particles and an air stream, turbulence, shear stress and collision. Each mechanism occurs to a different extent in most deagglomeration rigs.

Shear force fluidization occurs when a gas stream is passed over a powder source, contained in either a pocket or on an open surface. Powder agglomerates on the surface of the powder source experience reduced interparticle forces, as they are surrounded by fewer particles. Separation by shear force results in the transmission of both translational and rotational motion to the powder agglomerates as they are entrained by the gas stream. Collisions between powder agglomerates force the powder agglomerates to bounce, resulting in incipient fluidization. Powder agglomerates are separated from the bulk powder with high rotational velocities, for instance, in the vicinity of 1000 rev/s, generating Saffman lift forces that project the particles vertically. The high viscous shear stresses in the boundary layer close to the surface of the powder source magnify the vertical projection due to the Magnus force. This form of fluidization primarily affects powder agglomerates having diameters greater than 100 μm and is dependent on the velocity of the airflow around the powder agglomerates.

Shear force fluidization predominates in the majority of passive dry powder inhalers, i.e., inhalers in which the inspiratory flow is the sole source of energy for entraining the powder. Some inhalers use carriers, such as lactose, to carry smaller drug particles adhered to their surface. In such inhalers, although shear force fluidization dispenses carrier particles, the gas stream often flows directly through the powder source, rather than over it, resulting in the entrainment of large agglomerates of powder. This is referred to as “gas-assist” fluidization. Often, inhalers using “gas-assist” fluidization must provide a further stage of deagglomeration, since the entrained particles are not fine enough to escape impaction in the mouth and throat.

Particle collision is another important mechanism for the deagglomeration of powder agglomerates. Collisions can occur between powder agglomerates and between powder agglomerates and solid boundaries. Particle collisions with solid boundaries are usually promoted by introducing obstacles in the flow path, e.g. curved plates, where inertial impaction of particles will occur. For example, U.S. Pat. No. 2,865,370, issued to Gattone on Dec. 23, 1958, discloses a dispersing adaptor for use with disposable aerosol units wherein the carrier and drug powder particles entrained by gas-assist fluidization are discharged by the disposable aerosol units against a curved surface. Similarly, U.S. Pat. No. 4,940,051, issued to Lankinen on Jul. 10, 1990, discloses an inhalation device involving a curved baffle plate which deflects an aerosol discharge into an inhalation chamber. Furthermore, U.S. Pat. No. 6,427,688, issued to Ligotke et al. on Aug. 6, 2002, discloses a dry powder inhaler having a dispersion chamber containing at least one bead that assists in deagglomerating of drug particles. The beads roll, bounce, and collide repeatedly with drug particles on the chamber surfaces and on the beds. These devices also involve interparticle collisions, which are dependent on particle size, number concentration and particle-to-particle and gas-to-particle relative motion.

In the literature, turbulence is pointed out to be the principal factor in deagglomeration, without considering the detailed nature of turbulent fluid flow and its interaction with dispersed particles. Turbulence used for deagglomeration is typically produced by jets, grids and free shear layers. Exact analysis of the mechanics. involved in turbulence is difficult due to the complex nature of turbulence and the irregular particle shapes involved. It is normally assumed that deagglomeration happens when agglomerates of powders are buffeted by turbulent eddies that exert aerodynamic forces on the agglomerates and its individual particles. The magnitude of such forces mainly depends on turbulent scales.

In designing devices to deagglomerate powder agglomerates, the above described mechanisms may be used for reaching the highest fine powder fraction possible.

However, it should be noted that high fine particle fraction is itself not necessarily a good indicator of inhaler performance, since most dry powder inhalers deposit much of these fine particles on the walls of the extrathoracic region (from the mouth opening to the end of the trachea), giving losses and departure from the ideal delivery. Indeed, a more telling measure of inhaler performance is the amount of drug delivered past the mouth-throat region and into the lungs.

There is therefore a need in the market for a powder deagglomeration device and method that can achieve optimal delivery of powder to the lungs of a patient with relatively lower thresholds of mouth and throat powder deposition compared to known devices and methods, in a simple and efficient manner.

### SUMMARY OF THE INVENTION

According to the present invention, there is provided a device for deagglomerating powder agglomerates for inhalation, comprising:

a body having a chamber adapted for fluid circulation therethrough;

an inlet connected to the chamber and to a powder source for supplying the chamber with powder agglomerates entrained in a flow of gas, the powder agglomerates and the flow of gas defining a swirling fluid flow inside the chamber, the powder agglomerates being subjected to at least one of turbulence, shear force fluidizing, collisions with other ones of the powder agglomerates, and collisions with a surface of the chamber;

an outlet connected to the chamber for inhalation such that the swirling fluid flow in the chamber can exit from the chamber as a longitudinal fluid flow and secondary fluid flow, the longitudinal fluid flow being directed along a longitudinal axis of the outlet, and the secondary fluid flow being directed away from the longitudinal axis of the outlet; and

a mesh in the outlet for preventing powder agglomerates above a predetermined size from traversing the mesh, and for reducing the secondary fluid flow relative to the longitudinal fluid flow exiting from the chamber to thereby reduce powder deposition in a mouth and throat of a user.

Further in accordance with the present invention, there is provided a method for deagglomerating powder agglomerates for inhalation, comprising the steps of:

a) providing a body having a chamber adapted for fluid circulation therethrough;

b) supplying the chamber with powder agglomerates entrained in a flow of gas via an inlet connected to the chamber and to a powder source, the powder agglomerates and the flow of gas defining a swirling fluid flow inside the chamber, the powder agglomerates being subjected to at least one of turbulence, shear force fluidizing, collisions with other ones of the powder agglomerates, and collisions with a surface of the chamber;

c) connecting an outlet to the chamber for inhalation such that the swirling fluid flow in the chamber can exit from the chamber as a longitudinal fluid flow and secondary fluid flow, the longitudinal fluid flow being directed along a longitudinal axis of the outlet, and the secondary fluid flow being directed away from the longitudinal axis of the outlet; and

d) positioning a mesh in the outlet for preventing powder agglomerates above a predetermined size from traversing the mesh, and for reducing the secondary fluid flow relative to the longitudinal fluid flow exiting from the chamber to thereby reduce powder deposition in a mouth and throat of a user.

The invention as well as its numerous advantages will be better understood by reading of the following non-restrictive description of preferred embodiments made in reference to the appending drawings.

### BRIEF DESCRIPTION OF THE DRAWINGS

FIG. 1 is a top perspective view, partly exploded, of a deagglomeration device according to a preferred embodiment of the present invention.

FIG. 2 is a bottom perspective view of the deagglomeration device shown in FIG. 1.

FIG. 3 is perspective view of a shell of the deagglomeration device shown in FIG. 1, generally illustrating a position of the outlet with respect to the inlet.

FIG. 4 is another perspective view of the shell of the deagglomeration device shown in FIG. 3, illustrating a chamber of the shell.

FIG. 5 is a cross-section view taken along line V-V of the deaglomeration device shown in FIG. 2, illustrating the use of a mouthpiece.

FIG. 6 is a cross-section view of an deagglomeration device according to a second preferred embodiment of the present invention, illustrating the use of another type of mouthpiece.

### DESCRIPTION OF THE PREFERRED EMBODIMENTS

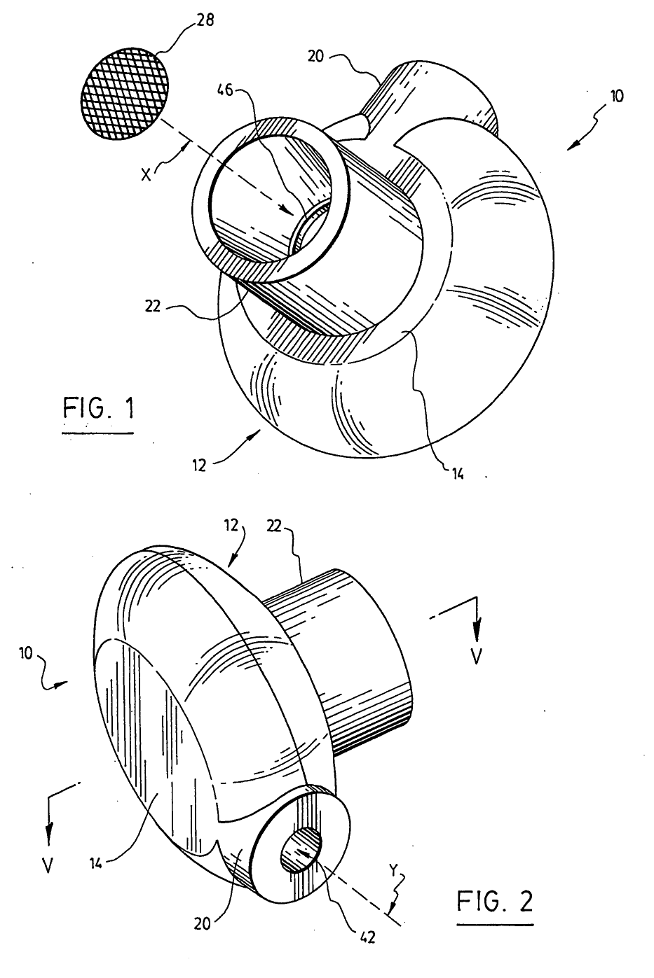

Referring to FIGS. 1 to 6, there is shown a deagglomeration device 10 according to a preferred embodiment of the present invention. The deagglomeration device 10 has a body 12 defining a chamber 40 adapted for fluid circulation therethrough. The device 10 has an inlet 20 connected to the chamber 40 and to a powder source (not shown) for supplying the chamber 40 with powder agglomerates entrained in a flow of gas. The powder agglomerates and the flow of gas define a swirling fluid flow inside the chamber 40. The powder agglomerates are subjected to at least one of turbulence, shear force fluidizing, collisions with other ones of the powder agglomerates, and collisions with a surface 41 of the chamber 40. The device 10 has an outlet 22 connected to the chamber 40 for inhalation such that the swirling fluid flow in the chamber 40 can exit from the chamber 40 as a longitudinal fluid flow and secondary fluid flow, the longitudinal fluid flow being directed along a longitudinal axis X of the outlet 22, and the secondary fluid flow being directed away from the longitudinal axis X of the outlet 22. The device also has a mesh 28 in the outlet 22 for preventing powder agglomerates above a predetermined size from traversing the mesh 28, and for reducing the secondary fluid flow relative to the longitudinal fluid flow exiting from the chamber 40 to thereby reduce powder deposition in the mouth and throat of a user.

Preferably, the mesh 28 is positioned near a base of the outlet 22 that is adjacent to the surface 41 of the chamber 40 so that most of the powder agglomerates in the chamber 40 collide with the mesh 28 at an oblique angle to assist in deagglomerating of the powder agglomerates inside the chamber 40. It is to be understood that the exact position of the mesh 28 in the outlet 22 can be varied. Optimal results for deagglomeration are achieved when the surface of mesh 28 is positioned perpendicular to the longitudinal axis of the exit channel 46 of the swirling flow inside the chamber 40. As shown for example in FIG. 4, the surface of mesh 28 is preferably tangential with the adjacent surface 41 of the chamber 40. Obviously, it should be noted that the farther away that the mesh 28 is positioned from the base of the outlet 22, then the less effective it will be in assisting in the deagglomeration of particles. The mesh 28 will nevertheless maintain its property of reducing powder deposition in the mouth and throat of the user whatever its location in the outlet 22. Preferably, the mesh 28 has a pore size of less than 250 μm, and more particularly, the pore size of the mesh 28 may range between 30 to 150 μm.

Preferably, the chamber 40 is a cyclone chamber having a disc-shaped portion 14 similarly to the body 12. Such chamber 40 does not present any sharp edges. More precisely, the peripheral surface of the chamber 40 has smooth round edges.

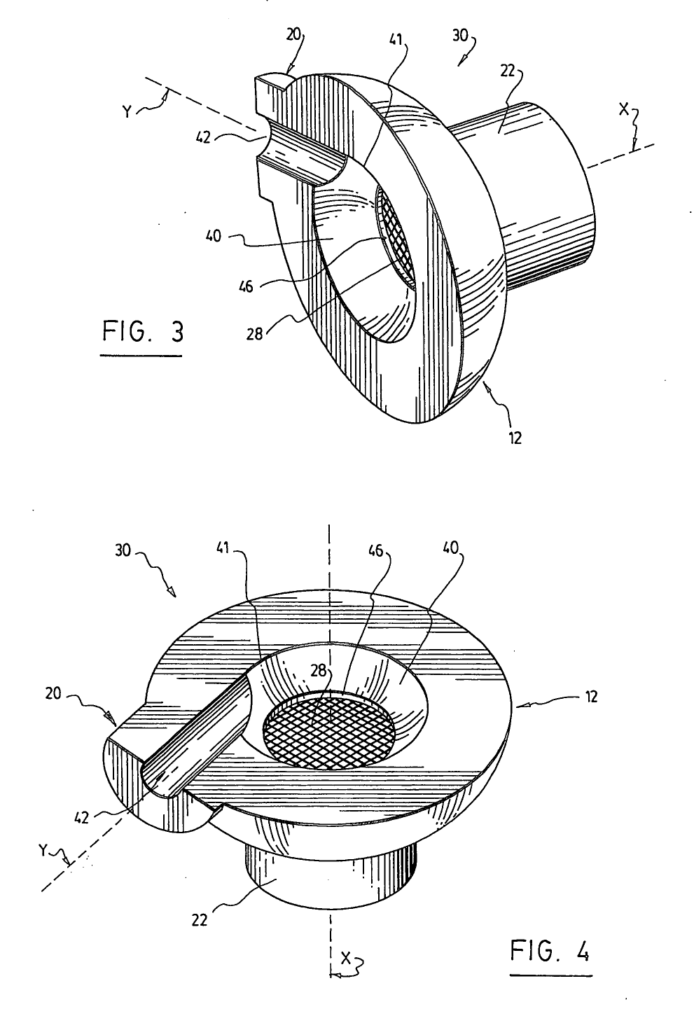

Referring to FIGS. 3 and 4, the body 12 is shown divided into two shells, one of which is shown at 30. The separation plane between the two shells is perpendicular to the outlet 22. The two shells are preferably symmetrically identical, except for the outlet 22 on the shell 30, which is not present on the other shell.

Preferably, the inlet 20 has a fluidizing channel 42 that merges tangentially with the chamber 40. The outlet 22, on the other hand, may protrude axially from the chamber 40. The outlet 22 defines a channel 46 that is preferably perpendicular to the chamber 40. In other words, the inlet 20 has a longitudinal axis Y that is perpendicular with respect to the longitudinal axis X of the outlet 22. The longitudinal axis Y of the inlet 20 is offset from the longitudinal axis X of the outlet 22 so that an inner surface at a base of the inlet 20 is tangential with respect to the surface 41 of the chamber 40. The mesh 28, as shown in FIG. 4, is disposed across the channel 46, so as to impede particles that are larger than a predetermined size from exiting from the chamber 40. Preferably, the inlet 20 has an internal diameter of 5 to 7 mm and the outlet 22 has an internal diameter of 8 to 12 mm.

The above configuration can obviously be subject to many changes as those skilled in the art will understand. Indeed, the exact orientation and position of the inlet 20 and outlet 22 with respect to one another may be varied. Importantly, it should be noted that the inlet 20 and outlet 22 do not necessarily have to be perpendicular to one another. Indeed, the purpose, shape and orientation of the inlet 20, outlet 22 is to form an adequate swirling fluid flow inside the chamber 40.

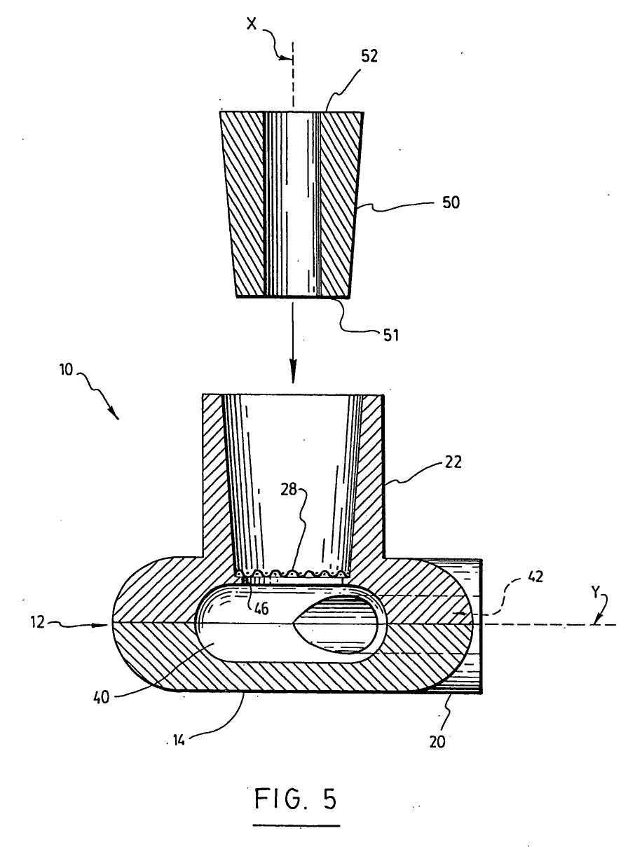

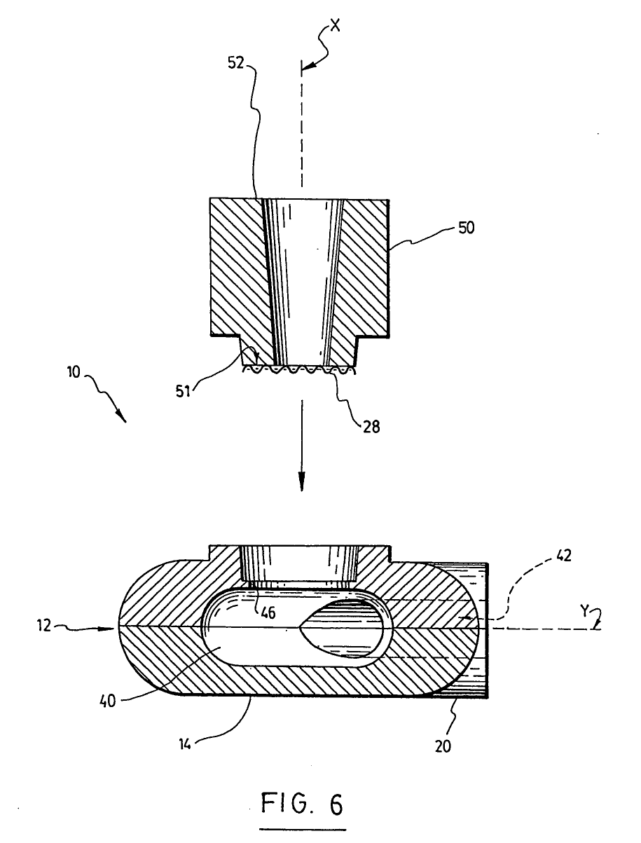

Referring to FIGS. 5 and 6, the device 10 may further include a mouthpiece 50 with a first end 51 being connectable to the outlet 22 and a second end 52 being insertable in the mouth of the user. The mouthpiece 50 may include a straight diffuser with a 13 to 15 degrees deflection. The mouthpiece 50 may have an internal diameter of 15 to 25 mm and a length of 5 to 25 mm. As shown in FIG. 5, the mesh 28 may be permanently located at the base of the outlet 22 while the mouthpiece 50 may be connected separately to the outlet 22. In the embodiment shown in FIG. 6, the mesh 28 is shown connected to the first end 51 of the mouthpiece 50 before being connected to the device 10.

Now that the configuration of the deagglomeration device 10 has been described, a method of operation of the deagglomeration device 10 will be described.

Prior to the use of the deagglomeration device 10 for deagglomeration of powder agglomerates for inhalation, the inlet 20 is connected to a powder source such as a powder capsule (not shown) so that powder and air can enter through channel 42 when the user inhales from the outlet 22. It should be understood by those skilled in the art that many different powder sources may be used and that the manner of introducing the air flow and powder may be varied.

As mentioned above, a mouthpiece 50 may be mounted to the outlet 22. Alternatively, the outlet 22 can directly serve as a suction end by the user.

During operation of the deagglomeration device 10, a pressure drop is created between the outlet 22 and the chamber 40. This is typically performed by a suction exerted by the user at the outlet 22. The pressure drop created in the chamber 40 is compensated by an inlet of a fluid (e.g., air) through the channel 42 of the inlet 20. Preferably, the inlet 20 and the powder source are open to the ambient air, and air will be sucked in through the channel 42 because of the pressure drop in the chamber 40. As air flows into the chamber 40 through the channel 42, powder from the powder source also comes in through the same channel 42 and then is entrained into the chamber 40.

In an alternative embodiment (not shown), the powder source may be connected perpendicularly to the channel 42 of the inlet 20. The mergence of the air flow with the powder will then create a shear force fluidization of the powder agglomerates, causing a certain level of deagglomeration.

A swirling turbulent motion is caused in the chamber 40 by the tangential position of the inlet portion 20 with respect to the chamber 40, and by the central position of the outlet 22. The turbulent motion will cause deagglomeration of agglomerates by the various forces it involves, and will also cause powder agglomerates to collide with one another, thereby further causing deagglomeration. Moreover, further collision will occur between the surface of the chamber 40 and the powder agglomerates.

As the powder agglomerates reach the outlet 22 and are sucked out therefrom, the mesh 28 represents an obstacle that prevents agglomerates beyond a predetermined size from exiting the chamber 40. Therefore, the mesh 28 must be sized in order to selectively filter out powder agglomerates above a given size. These powder agglomerates will be rebounded to the chamber 40 and, by the swirling turbulence in the chamber 40, will be further deagglomerated by colliding with other powder agglomerates and/or colliding with the surface of the chamber 40 or with the surface of the mesh 28 if it is placed near the base of the outlet 22, or simply by the forces of turbulence. The other function of the mesh 28 is to reduce the secondary fluid flow relative to the longitudinal fluid flow exiting from the chamber 40 so that powder deposition in the mouth and throat of a user is also reduced.

Various configurations are contemplated for the use of the deagglomeration device 10. For instance, a powder source (not shown) connected to the inlet 20 can be a dosage-controlling mechanism that will ensure that each inhalation involves a predetermined amount of powder. Also, it is possible to cause the pressure drop between the chamber 40 and the outlet 22 by injecting a fluid (e.g. air) through the inlet 20.

The fine powder fraction reached by the deagglomeration device 10 is generally above the fine powder fraction reached by marketed inhalers and it has the additional advantage of reducing the powder deposition in the mouth and throat of the user. Such results can be obtained using the following parameters for the deagglomeration device 10:

Flow rate through cascade impactor: 60 LPM

Drug used: Micronized mixture of ciprofloxacin, phospholipids and lactose; also Ventodisk® powder (mixture of lactose and salbutamol sulphate)

Inlet air pressure: atmospheric

Inner diameter of the fluidizing channel: 6 mm

Mesh used: 400# (38 μm)

Fine powder fraction reached:

56%-87% by the deagglomeration device 10

Fine powder fraction reached using similar parameters with other inhalers:

15%-36% by other marketed inhalers

36% by Ventodisk®

Further optimizations and experimental tests have been done for an inhaler according to the present invention to reduce the pressure resistance and raise the fraction delivered distal to the mouth-throat at a flow rate 30 LPM and 60 LPM. The addition of the mesh 28 of a certain size in the inhaler has been found to reduce mouth-throat deposition to the lowest possible levels achievable with any inhaler i.e. the mesh reduces mouth-throat deposition to levels seen when aerosols are inhaled from ambient air with a straight tube.

The excellent deagglomeration abilities of the inhaler are demonstrated by its high fine particle fraction (e.g. >70% at an inhalation flow rate of 60 L/min.). With the present inhaler and mouthpiece design, experiments have show that at an inhalation flow rate of 60 L/min, a total of 70% of the dose loaded into the inhaler is delivered past a proper representation of mouth-throat when a fine mesh is used in the inhaler. Without the fine mesh in place, the dose delivered past the mouth-throat drops to 46% indicating the tremendous utility of the mesh in reducing mouth-throat deposition. The reason for the reduction in mouth-throat deposition caused by the mesh is probably related to the mesh causing a dramatic reduction in secondary, swirling flow velocities entering the mouth.

Although preferred embodiments of the present invention have been described in detail herein and illustrated in the accompanying drawings, it is to be understood that the invention is not limited to these precise embodiments and that various changes and modifications may be effected therein without departing from the scope or spirit of the present invention.

1. A device for deagglomerating powder agglomerates for inhalation, comprising:
a body having a chamber adapted for fluid circulation therethrough;
an inlet connected to the chamber and to a powder source for supplying the chamber with powder agglomerates entrained in a flow of gas, the powder agglomerates and the flow of gas defining a swirling fluid flow inside the chamber, the powder agglomerates being subjected to at least one of turbulence, shear force fluidizing, collisions with other ones of the powder agglomerates, and collisions with a surface of the chamber;
an outlet connected to the chamber for inhalation such that the swirling fluid flow in the chamber can exit from the chamber as a longitudinal fluid flow and secondary fluid flow, the longitudinal fluid flow being directed along a longitudinal axis of the outlet, and the secondary fluid flow being directed away from the longitudinal axis of the outlet; and
a mesh in the outlet for preventing powder agglomerates above a predetermined size from traversing the mesh, and for reducing the secondary fluid flow relative to the longitudinal fluid flow exiting from the chamber to thereby reduce powder deposition in a mouth and throat of a user.

2. The device according to claim 1, wherein the mesh is positioned near a base of the outlet that is adjacent to the surface of the chamber so that most of the powder agglomerates in the chamber collide with the mesh at an oblique angle to assist in deagglomerating of the powder agglomerates inside the chamber.

3. The device according to claim 1, wherein the chamber is a cyclone chamber having a disc-shaped portion, the inlet having a longitudinal axis that is perpendicular with respect to the longitudinal axis of the outlet, the longitudinal axis of the inlet being offset from the longitudinal axis of the outlet so that an inner surface at a base of the inlet is tangential with respect to the surface of the chamber.

4. The device according to claim 2, wherein the mesh has a pore size of less than 250 μm.

5. The device according to claim 4, wherein the pore size of the mesh ranges between 30 to 150 μm.

6. The device according to claim 2, wherein the inlet has an internal diameter of 5 to 7 mm and the outlet has an internal diameter of 8 to 12 mm.

7. The device according to claim 1, further comprising a mouthpiece having a first end being connectable to the outlet and a second end being insertable in the mouth of the user.

8. The device according to claim 7, wherein the mesh is connected to the first end of the mouthpiece.

9. The device according to claim 7, wherein the mouthpiece includes a straight diffuser with a 13 to 15 degrees deflection, and has an internal diameter of 15 to 25 mm and a length of 5 to 25 mm.

10. A method for deagglomerating powder agglomerates for inhalation, comprising the steps of:
a) providing a body having a chamber adapted for fluid circulation therethrough;
b) supplying the chamber with powder agglomerates entrained in a flow of gas via an inlet connected to the chamber and to a powder source, the powder agglomerates and the flow of gas defining a swirling fluid flow inside the chamber, the powder agglomerates being subjected to at least one of turbulence, shear force fluidizing, collisions with other ones of the powder agglomerates, and collisions with a surface of the chamber;
c) connecting an outlet to the chamber for inhalation such that the swirling fluid flow in the chamber can exit from the chamber as a longitudinal fluid flow and secondary fluid flow, the longitudinal fluid flow being directed along a longitudinal axis of the outlet, and the secondary fluid flow being directed away from the longitudinal axis of the outlet; and
d) positioning a mesh in the outlet for preventing powder agglomerates above a predetermined size from traversing the mesh, and for reducing the secondary fluid flow relative to the longitudinal fluid flow exiting from the chamber to thereby reduce powder deposition in a mouth and throat of a user.

11. The method according to claim 10, wherein step d) comprises the step of positioning the mesh near a base of the outlet that is adjacent to the surface of the chamber so that most of the powder agglomerates in the chamber collide with the mesh at an oblique angle to assist in deagglomerating of the powder agglomerates inside the chamber.

12. The method according to claim 1, wherein step a) the chamber is a cyclone chamber having a disc-shaped portion, the inlet having a longitudinal axis that is perpendicular with respect to the longitudinal axis of the outlet, the longitudinal axis of the inlet being offset from the longitudinal axis of the outlet so that an inner surface at a base of the inlet is tangential with respect to the surface of the chamber.

13. The method according to claim 11, wherein step d) the mesh has a pore size of less than 250 μm.

14. The method according to claim 13, wherein step d) the pore size of the mesh ranges between 30 to 150 μm.

15. The method according to claim 11, wherein in step b) the inlet has an internal diameter of 5 to 7 mm and in step c) the outlet has an internal diameter of 8 to 12 mm.

16. The method according to claim 10, further comprising the step of e) providing a mouthpiece having a first end being connectable to the outlet and a second end being insertable in the mouth of the user.

17. The method according to claim 16, wherein step e) the mesh is connected to the first end of the mouthpiece.

18. The method according to claim 16, wherein step e) the mouthpiece includes a straight diffuser with a 13 to 15 degrees deflection, and has an internal diameter of 15 to 25 mm and a length of 5 to 25 mm.
